(19) United States
(12) Patent Application Publication
(10) Pub. No.: US 2005/0058767 A1
(43) Pub. Date: Mar. 17, 2005

(54) Process for accelerating recovery from trauma by using apoptosis-mimicking synthetic or natural entities

(76) Inventors: Anthony E Bolton, Bakeswell (GB); Arkady Mandel, North York (CA)

Correspondence Address: FOLEY & LARDNER LLP;THREE PALO ALTO SQUARE, 3000 EL CAMINO REAL, SUITE 100, PALO ALTO, CA, 94306, US

(21) Appl. No.: 10/490,001

(22) PCT Filed: Sep. 13, 2002
(86) PCT No.: PCT/CA02/01396

Related U.S. Application Data
(60) Provisional application No. 60/323,502, filed on Sep. 18, 2001.

(51) Int. Cl. B05D 3/00; A61L 2/00

(52) U.S. Cl. 427/2.1

(57) ABSTRACT

Mammalian patients suffering from post-wounding trauma, or about to or likely to suffer such trauma (by surgical treatment, or by suffering unanticipated accidental injuries, battle injuries or the like) are treated to lessen the severity of or accelerate the recovery from such subsequently sustained trauma, by administering to the patient immune system-modifying entities, each comprising a body of a size similar to an apoptotic mammalian cell or apoptotic body, and having exposed on its surface phos-pho-amino acid groups, the entities being capable of being taken up by cells of the patient=s immune system with accompanying beneficial effects including inhibition of pro-inflammatory cytokines and/or promotion of anti-inflammatory cytokines. The entities may be phosphatidylserine-presenting liposomes or natural products, beads carrying phospho-serine or phosphatidylserine side groups, apoptotic cells or apoptotic bodies, generally of size 50 nanometers to 500 microns, and administered to contact a patient=s immune system (e.g. intramuscularly) in amounts which affect but do not overwhelm the patient=s immune system.

### FIELD OF THE INVENTION

This invention relates to therapeutic compositions and uses thereof in medical treatments and prophylaxis to lessen the effects of adverse medical conditions. More specifically, it relates to acceleration of recovery of a patient from the physical trauma of surgery and other wounding and injury conditions, and to methods of pre-conditioning the mammalian body so as better to withstand such physical trauma.

### BACKGROUND OF THE INVENTION

There is a continuing need to shorten the hospital stay of patients undergoing surgical procedures, which effectively means accelerating the rate of recovery of a patient from the trauma of surgery or other injuries. This applies both to patients undergoing pre-scheduled or elective surgery, and to patients undergoing surgery as a result of accidental injury or treatment of an unforeseen medical emergency. Both for the comfort and rapid recovery of the patient, and for the benefit of health care economics, it is desirable to be able to accelerate the rate of recovery of a patient from trauma after surgery or after suffering an accidental wound or injury.

It would also be desirable to be able to pre-condition a patient scheduled to undergo surgery, so that the patient would be better able to withstand the trauma associated with surgery, to lead to a more rapid recovery from trauma afterwards. It would also be advantageous to be able to precondition persons at risk of sustaining injury (battle troops, rescue personnel and the like) to enable them to recover more rapidly from such trauma.

### SUMMARY OF THE INVENTION

The present invention is based upon the novel appreciation of the role played by the up-regulation of anti-inflammatory cytokines and/or the down regulation of inflammatory cytokines in a patient's body, and by improved endothelial function, on the mammalian body's process of recovery from the trauma of surgery and other wounds. The natural process of apoptosis (programmed cell death) leads to the upregulation of anti-inflammatory cytokines and the down-regulation of inflammatory cytokines in the mammalian body, as well as improvements in endothelial function. The present invention provides a process of accelerating the recovery of a patient from the trauma of wounds (surgical or accidental), and a process of pre-conditioning to accelerate the recovery from subsequently experienced such trauma, which mimics the apoptosis process of the mammalian body and takes advantage of the beneficial effects flowing from apoptosis in vivo, to effect such processes.

According to one aspect of the present invention, there is provided a process of treating a mammalian patient suffering from post-wounding trauma, or treating a mammalian patient about to or likely to suffer such trauma (by surgical treatment, or by suffering unanticipated accidental injuries, battle injuries or the like) to lessen the severity of or accelerate the recovery from such subsequently sustained trauma, comprising administering to the patient an effective immune system modifying amount of immune system-modifying entities, each comprising a body of a size similar to an apoptotic mammalian cell or apoptotic body, and having exposed on its surface phospho-amino acid groups, the entities being capable of being taken up by cells of the patient's immune system with accompanying beneficial effects including inhibition of pro-inflammatory cytokines and/or promotion of anti-inflammatory cytokines.

### THE PREFERRED EMBODIMENTS

One category of such entities is natural biological vesicles presenting phosphatidylserine (PS) on an outer membrane surface. Such vesicles, upon administration to a mammalian patient, will mimic the apoptosis process with consequent down-regulation of pro-inflammatory cytokines and/or upregulation of anti-inflammatory cytokines. Immune cells can engulf the entities as the PS groups on the membranes thereof interact with the PS receptors in an in vivo process resembling apoptosis, with consequent down-regulation of pro-inflammatory cytokines and/or upregulation of anti-inflammatory cytokines. Natural biological vesicles presenting PS on an external membrane surface, of particular interest in this embodiment of the present invention, include the following:

Exosomes, which are microvesicles exfoliated from cultured cells, and may also be produced in vivo, e.g. during maturation of reticulocytes (see Trams et.al., Biochimica et. Physica Acta, 645 (1981) 63-70; and also Johnstone, Biochem. Cell. Biol.,70 (1982) 179-190);

Prostasomes, which are vesicular extracellular organelles found in seminal plasma (see Rooney et. al., J. Exp. Med. 177, May 1993, 1409-1420);

Spontaneous or induced shed membrane vesicles, i.e. membrane vesicles shed from cells as a result of inducement using detergents such as lysophasphatidylcholine, or spontaneously (see Ferber et. al., Biochimica et Biophysica Acta, 595 (1980) 244-256; also Emerson et. al., The Journal of Immunology, 127 (2), August 1981, 482-486);

Procoagulant bound to plasma membrane vesicles, i.e. thromboplastin-like activity associated with membrane vesicles, found for example in bronchoalveolar lavage fluid and derived from alveolar macrophages (see Lyberg et. al., Eur. Respir. J., 3 (1990), 61-67);

Inside out red blood cell ghosts, which express PS on the outer surface, and sickle cell red blood cells which express PS on the surface as part of the pathology (see Schroit et. al., Biol. Cell 51 (1984) 227-238);

Erythrocytes with lost phospholipid asymmetry, i.e. erythrocytes with randomized, symmetric transbilayer distribution of phospholipids; these can be produced, for example, by elevating intracellular Ca⁺⁺ levels (see Pradham et. al. Molecular Membrane Biology 11 (1994) 181-188);

Activated platelets, platelets with pro-coagulant activity, which are associated with re-orientation of PS from the inner to the outer leaflet of the platelet membrane bilayer (see Bevers et. al., Biochimica et Biophysica Acta, 736: (1983) 57-66);

Platelet derived microparticles, which are membranous vesicles or microparticles shed from platelet membranes following platelet activation (see Gilbert et. al., The Journal of Biological Chemistry, 266 No.26, Sep. 16, 1991, 17261-17268).

Such natural biological vesicles can be used for administration to patients about to suffer trauma, e.g. patients about to undergo surgery or at high risk of suffering trauma from wounds as a result of imminent battle action, natural disaster etc., and will precondition the patient's body so as to accelerate the recovery from such trauma. They will also have the effect of accelerating the recovery of a patient from trauma when administered to an already traumatized patient.

Another category of such entities, the use of which in treatment of trauma and preconditioning against trauma constitutes another embodiment of the present invention, is biocompatible synthetic entities comprising:
a three-dimensional head portion of size in its largest dimension of from 50 nanometers to 500 microns;
a plurality of tail portions bonded to each said head portion, the tail portions having:
   phospho-amino acid end groups capable of interaction with receptors on antigen-presenting cells,
   and chemical spacer groups of at least 3 linear carbon atoms, the spacer groups being bonded at their proximal ends to the respective head portion, and at their distal ends to the phosphate of the phospho-amino acid group.

The phospho-amino acid groups forming the end groups of the entities used in this embodiment of the invention have the general formula: 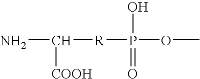
in which R represents C1-C4 straight chain or branched alkylene, alkylene-oxy, alkylene-thio, alkylene-amine, phenyl, iodo-substituted phenyl, and 5-membered N-heterocyclic groups, with the proviso that they interact with appropriate receptors on antigen-presenting cells.

Preferred phospho-amino acid groups in the compositions used in this embodiment of the present invention are phospho-serine and phosphothreonine groups, with the most preferred being phosphoserine of formula:

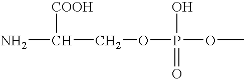

In a particularly preferred embodiment, the tail portions of the derivatized beads have the chemical formula: 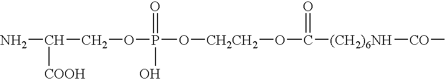
the amide end group being bonded to the head portion surface.

The term “beads” as used herein is intended to mean substantially any biocompatible body, solid, semisolid or hollow, shape-retaining and typically but not exclusively spheroidal, cylindrical, ellipsoidal including oblate and prolate spheroidal, serpentine, reniform, etc., and from about 50 nanometers to about 500 microns in diameter. They may be flexible or rigid. Preferred materials for their composition are polyrnethylmethacrylate, polacrylate, polymethacrylate, glass, polystyrene, polyethylene, polypropylene and the like, of a grade approved for administration to mammalian patients.

The phospho-amino acid end groups in entities used in this embodiment of the invention may be the distal end group of a phospholipid, the proximal end of which is attached to a body. These include particles, granules, microspheres or beads of biocompatible materials, natural or synthetic, such as polyethylene glycol, polyvinylprrolidone, polystyrene, etc., polysaccharides such as hydroxethyl starch, hydroxyethylcellulose, agarose and the like, as commonly used in the pharmaceutical industry. Some such suitable substances for derivatization to attach the PS and, in the case of agarose, with PS attached, are commercially available, e.g. from Polysciences, Inc. 400 Valley Road, Warrington, Pa. 18976, or from Sigma Aldrich Fine Chemicals. The beads may be solid or hollow, or filled with biocompatible material. They are modified as required so that they carry PS molecules on their surfaces.

Such phospho-amino acid carrying entities can be used for administration to patients about to suffer trauma involving wounds, e.g. patients about to undergo surgery or at high risk of suffering a wound as a result of imminent battle action, natural disaster etc., and will precondition the patient's body so as to accelerate the recovery from such subsequently encountered trauma. They will also have the effect of accelerating the recovery of a patient when administered to an already traumatized patient.

A further category of entities for use in another embodiment of the invention is liposomes of the appropriate sizes referred to above, i.e., sizes resembling those of apoptotic mammalian cells or apoptotic bodies, and which have surface PS molecules. As a phospholipid, PS can form the membrane of a liposome, either as the sole constituent of the membrane or as a major or minor component thereof, with other phospholipids and/or membrane forming materials. Liposomes, or lipid vesicles, are sealed sacs, in the micron or sub-micron range, the walls of which consist of layers of suitable amphiphiles. They normally contain an aqueous medium.

Phospholipids are amphiphilic molecules (i.e. an amphiphiles), meaning that the compound comprises molecules having a polar water-soluble group attached to a water-insoluble hydrocarbon chain. The amphiphiles serving as the layers of the matrix have defined polar and apolar regions. The amphiphiles can include naturally occurring lipids such as PS, phosphatidylethanolamine, phosphatidylinositol, phosphatidylcholine, cholesterol, ceramides and sphingomyelin, used alone or in admixture with one another. They can be synthetic compounds such as polyoxyethylene alkylethers, polyoxyethylene alkylesters and saccharosediesters.

The present invention contemplates the use, not only of those liposomes having PS as a membrane constituent, but also liposomes having non-PS membrane substituent but which carry on their external surface molecules of PS, e.g., chemically attached by chemical modification of the liposome surface, making the PS available for subsequent interaction with components of the patient recipient's immune system.

Preferred are liposomes constituted to the extent of 50%-100% by weight of phosphatidylserine (PS), the balance being phosphatidylcholine (PC) or other such biologically acceptable phospholipid(s). More preferred are liposomes constituted by PS to the extent of 65%-90% by weight. They are prepared from mixtures of the appropriate amounts of phospholipids as starting materials, by known methods.

Methods of preparing liposomes of the appropriate size are known in the art and do not form part of this invention. Reference may be made to various textbooks and literature articles on the subject, for example the review article “Liposomes as Pharmaceutical Dosage Forms” by Yechezkel Barenholz and Daan J. A. Chromeline, and literature cited therein, for example New, R. C., “Liposomes: A Practical Approach;” IRL Press at Oxford University Press, Oxford, England (1990), and Nassander, U. K., et al., In: “Biodegradable Polymers as Drug Delivery Systems” (M. Chasin and R. Langer, eds.) Marcel Dekker Inc., New York 1990, pages 261-338.

Such PS-carrying liposomes can be used for administration to patients about to suffer trauma involving wounds, e.g. patients about to undergo surgery or at high risk of suffering a wound as a result of imminent battle action, natural disaster etc., and will precondition the patient's body so as to accelerate the recovery of such subsequently encountered trauma. They will also have the effect of accelerating the recovery of a patient when administered to an already traumatized patient

A further category of entities for use in another preferred embodiment of the present invention is apoptotic cells and apoptotic bodies themselves. “Apoptotic cells” and “apoptotic bodies,” as the terms are used herein, means cells and cell bodies which exhibit one or more of the following apoptosis-characterizing features: surface exposure of phosphatidylserine, as detected by standard, accepted methods of detection such as Annexin V staining; alterations in mitochondrial membrane permeability measured by standard, accepted methods (e.g. Salvioli, S., Ardizzoni, A., Franceschi, C. Cossarizza, A. (1997) “JC-1, but not DiOC6(3) or Rhodamine 123, is a Reliable Fluorescent Probe to assess Delta Psi Changes in Intact Cells: Implications for Studies on Mitochondrial Functionality during Apoptosis,” FEBS Letters 411: 77-82]; evidence of DNA fragmentation such as the appearance of DNA laddering on agarose gel electrophoresis following extraction of DNA from the cells [Teiger, E., Dam, T. V., Richard, L., Wisnewsky, C., Tea, B. S., Gaboury, L., Tremblay, J., Schwartz, K. and Hamet, P. (1996) “Apoptosis in Pressure Overload-induced Heart Hypertrophy in the Rat,” Journal of Clinical Investigation 97; 2891-2897], or by in situ labeling (see Gavrieli et al., 1992, referenced above).

The apoptotic cells and/or apoptotic bodies for use in this embodiment of the invention are prepared ex vivo from mammalian cells that are compatible with those of the mammalian patient to whom they are to be administered. They can be prepared from substantially any type of mammalian cell including cultured cell lines. Preferably they are prepared from a cell type derived from the mammalian patient's own body or from an established cell line. More preferably they are prepared from white blood cells of blood compatible with that of the mammalian patient, more preferably from the patient's own white blood cell and even more preferably from the patient's own T lymphocytes. Even more preferably they are prepared from an established cell line. The apoptotic cells and/or apoptotic bodies are prepared extracorporeally prior to administration to the patient. Thus, in one embodiment, an aliquot of the patient's blood may be withdrawn, e.g. by venipuncture, and at least a portion of the white cells thereof subjected extracorporeally to apoptosis inducing conditions.

A variety of methods of inducing apoptosis in mammalian cells, so as to create apoptotic cells and apoptotic bodies, are known in the art and essentially any of these can be adopted in preparing apoptotic bodies for use in the present invention. One such method is the subjection of the cells to ionizing radiation (Y-rays, x-rays, etc.) and/or non-ionizing electromagnetic radiation including ultraviolet light. Apoptosis can be induced by subjecting cells to ultrasound.

Another method is the treatment of the cells with drugs such as non-specific protein kinase inhibitors as exemplified by staurosporine (see Bombeli, Karsan, Tait and Hirlan, (1997) “Apoptotic Vascular Endothelial Cells Become Procoagulant”, Blood, Vol. 89:2429-2442). Also, certain chemotherapeutic agents used for the treatment of malignant tumours induce apoptosis, for example adriamycin, as can statin drugs (3-hydroxy-3methylglutaryl coenzyme A reductase inhibitors) [Guijarro C, Blanco-Colio L M, Ortego M, Alonso C, Ortiz A, Plaza J J, Diaz C, Hernandez G, Edigo J (1998), “3-hydroxy-3methylglutaryl coenzyme A reductase and isoprenylation inhibitors induce apoptosis of vascular smooth muscle in culture,” Circulation Research 83: 490-500] and colcicine [Suzuki Y (1998)”, “Cell death, phagocytosis and neurogenesis in mouse olfactory epithelium and vomeronasal organ after colcicine treatment,” Annals of the New York Academy of Sciences 855: 252-254]. The use of ligands for death receptors on cells, such as Fas-ligand, will be apparent for inducing apoptosis from the discussion of apoptosis above.

Yet another method is the application of oxidative stress to cells extracorporeally (see for example Buttke and Sandstrom (1994) “Oxidative Stress as a Mediator of Apoptosis,” Immunology Today, Vol. 15:7-10). This can be achieved by treating the cells, in suspension, with chemical oxidizing agents such as hydrogen peroxide, other peroxides and hydroperoxides, ozone, permanganates, periodates, and the like. Biologically acceptable such oxidizing agents are preferably used, so as to reduce potential problems associated with residues and contaminations of the apoptotic cells and apoptotic bodies so formed.

This embodiment of the use of the present invention is not restricted to any particular method of producing apoptotic cells and apoptotic bodies, for use herein, and any suitable, known process can be used.

Methods for the detection and quantitation of apoptosis can be used to determine the presence and level of apoptosis in the preparation to be administered to the patient in the present invention. At least one of the methods from those described in the introduction above should be used to confirm the level of apoptosis achieved prior to administration. They are suitably purified prior to use, by methods known in the art, such as differential centrifugation.

One preferred process of preparing apoptotic cells and apoptotic bodies for use in the present invention involves the culture of cells from the patient, or a compatible mammalian cell line. The cultured cells may then be treated to induce apoptosis and to create apoptotic cells and/or apoptotic bodies therein. The cells, suspended in the patient's plasma or another suitable suspension medium, such as saline or a balanced mammalian cell culture medium, can then be administered as indicated below. The numbers of apoptotic cells and/or bodies can be determined by published methods available in the scientific literature on the subject including the above-reference articles. The numbers of such apoptotic cells and/or apoptotic bodies required for administration to the patient to obtain the required clinical benefit will vary depending on the source of cells, the patient's condition, the age and weight of the patient and other relevant factors which are readily determinable by the attending clinician.

The successful application of the process of the present invention may be manifested in several ways, individually or collectively. The patient may manifest accelerated rate of wound healing, and/or more rapid decline of elevated body temperatures resulting from inflammatory cytokine action and fever as a result of wounding. In addition or in the alternative, the patient may evidence a more rapid recovery of joint mobility, e.g. following orthopedic surgery to replace or to repair a defective body joint (knee, hip, shoulder, etc). A greater survival rate of seriously injured patients is to be anticipated as a result of the use of the present invention. As a result, the duration of the hospital stay for the patient can be significantly reduced.

Another common manifestation of patients obliged to spend long periods in bed as a result of trauma from injury is the development of medical ulcers (decubitus or pressure ulcers). The processes of the present invention are indicated for acceleration of the healing of such ulcers, and indeed for treating and accelerating the healing of mammalian ulcers in general, and thereby further contributing to the shortening of the duration of a patient's hospital stay.

The sizes of the immune modifying entities used in the invention is such that they will be taken up by cells of the patient's immune system in an apoptosis-mimicking fashion. In general, whatever type of entity is chosen, this means a size from about 50 nanometers to about 500 microns, more preferably from about 50 nanometers to about 500 nanometers.

The entities used in the process of the invention may be administered to the patient by any suitable means which brings them into operative contact with active ingredients of the patient's immune system. Preferably, the entities are constituted into a liquid suspension in a biocompatible liquid such as physiological saline and administered to the patient intra-arterially, intravenously or most preferably intramuscularly or subcutaneously.

A preferred manner of administering the entities to the patient is as a course of injections, administered daily, several times per week, weekly or monthly to the patient, over a period ranging from a week to several months. The frequency and duration of the course of the administration is likely to vary widely from patient to patient, and according to the severity of the trauma being treated or against which the patient is to be preconditioned. Its design and optimization is well within the skill of the attending physician.

The quantities of entities to be administered will vary quite widely depending on the severity of the trauma it is intended to treat or against which it is intended to precondition, and on the identity and characteristics of the patient. It is important that the effective amount of entities is non-toxic to the patient, and is not so large as to overwhelm the immune system.

When using intra-arterial, intravenous, subcutaneous or intramuscular administration of a liquid suspension of entities, it is preferred to administer, for each dose, from about 0.1-50 ml of liquid, containing an amount of entities generally equivalent to 1.0%-1000% of the nunber of cells normally found in an equivalent volume of whole blood or the number of apoptotic bodies that can be generated from them. Generally, the number of synthetic entities administered per delivery to a human patient is suitably in the range from about 500 to about 20×10⁹, preferably 10,000 to about 2×10⁹, as indicated by pre-clinical studies. Since animal models may not be truly representative of required numbers on a simple multiple of body weight, in an immune system modifying scenario, useful human dosage amounts may also be found in the 500-20,000,000 range.

Since the synthetic entities are acting, in the process of the invention, as immune system modifiers, in the nature of a vaccine, the number of such bodies administered to an injection site for each administration is a more meaningful quantitation than the number or weight of synthetic entities per unit of patient body weight. For the same reason, effective amounts or numbers of synthetic entities for small animal use may not directly translate into effective amounts for larger mammals on a weight ratio basis.

The invention is further described, for illustrative purposes, in the following specific example.

### EXAMPLE

The invention can be demonstrated by experiments on laboratory rats, pretreating them with a course of injections of phosphatidylserine liposomes, surgically inserting temperature and heartbeat measuring probes into the pre-treated animals, and measuring their body temperature and other vital signs using the probes, as a measure of their recovery from the surgical major laparotomy required for insertion. The results are predictive of the effects on other mammals, including humans.

A total of 30 seven week old laboratory bred rats is separated into two of 15 animals each. Each animal of the test group A is administered, on day 1, day 2 and day 14, an intragluteal injection of 75% phosphatidylserine—25% phosphatidylcholine liposomes of size 100±20 nanometers, suspended in PBS, of volume 150 μL, each injection comprising 1,800,000 liposomes. Each animal of the control group B is similarly administered 50 μL of PBS containing no liposomes, on days 1, 2 and 14.

Four days after the completion of the injections, the animals are anaesthetized, and a telemetry probe is inserted surgically into the femoral artery of each animal. The telemetry probe (DATAQUEST LABPRO, from Data Sciences International) is a commercially available probe equipped with a radio transmitter, to permit heartbeat, systolic blood pressure, diastolic blood pressure and other signals to be received without further handling of the animals. An additional probe is surgically inserted into the peritoneal cavity of each animal, to measure body temperature.

Continuous daily recordings of body temperature, blood pressure and heart rate are made from each animal, for 10 days following the surgery. The group A test animals show a noticeably faster recovery of normal body temperature than the control group B, demonstrating a faster rate of wound healing and recovery from surgery in the test group.

All patents, patent applications, and publications previously cited above are herein incorporated by reference in their entirety.

1-9. (canceled)

10 A process for treating a mammalian patient suffering from post-wounding trauma, or treating a mammalian patient about to or likely to suffer such trauma to lessen the severity of or accelerate the recovery from such subsequently sustained trauma, which process comprises administering to the patient an effective immune system modifying amount of immune system-modifying entities, each comprising a body of a size similar to an apoptotic mammalian cell or apoptotic body, and having exposed on its surface phospho-amino acid groups, the entities being capable of being taken up by cells of the patient's immune system with accompanying beneficial effects including inhibition of pro-inflammatory cytokines and/or promotion of anti-inflammatory cytokines.

11 The process of claim 10, wherein said trauma is selected from the group consisting of surgical treatment, unanticipated accidental injuries, and battle injuries.

12 The process of claim 10, wherein said entities are phosphatidylserine (PS) carrying bodies.

13 The process of claim 12, wherein said PS carrying bodies are natural entities.

14 The process of claim 12, wherein said PS carrying bodies are synthetic beads.

15 The process of claim 12, wherein said PS carrying bodies are PS liposomes.

16 The process of claim 15, wherein said PS liposomes comprise from about 50% to about 100% PS by weight.

17 The process of claim 10, wherein each body has a diameter of from about 50 nanometers to about 500 microns.

18 The process of claim 10, wherein said administering is administered in a unit dose of from about 500 to 2,000,000 of said entities.

19 The process of claim 10, wherein said administering is administered in a unit does of from about 500-20×10⁹ of said entities.
